(19) United States
(12) Patent Application Publication
(10) Pub. No.: US 2025/0288450 A1
(43) Pub. Date: Sep. 18, 2025

(54) WASTE CAPTURING DEVICE

(71) Applicant: Sebastian J Heredia, Raleigh, NC (US)

(72) Inventor: Sebastian J Heredia, Raleigh, NC (US)

(21) Appl. No.: 19/226,687

(22) Filed: Jun. 3, 2025

Related U.S. Application Data
(63) Continuation-in-part of application No. 18/619,386, filed on Mar. 28, 2024.
(60) Provisional application No. 63/528,901, filed on Jul. 25, 2023.

(51) Int. Cl. A61F 5/442; A61M 1/00; A61M 3/02

(52) U.S. Cl. CPC A61F 5/442; A61M 1/604; A61M 3/0287

(57) ABSTRACT

A waste capturing device to use with a human in prone, or supine position. It provides air and water jets connected to water and air source to clean the genital area. This system improves on the ability to keep waste under control, especially in a bed.

### REFERENCE TO RELATED APPLICATIONS

This application is a continuation in part of U.S. non-provisional application Ser. No. 18/619,386 filed on Mar. 28, 2024 which claims priority of U.S. provisional application No. 63/528,901 filed on Jul. 25, 2023 and is a continuation in part of PCT application number PCT-US24-48323 filed on Sep. 25, 2024.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a bedpan system for capturing waste. In particular it relates to a bedpan system which connects to vacuum, water and air sources, such as one normally finds in hospital settings.

### Description of Related Art

The use of bed pans is universal in hospitals for use with patients that can't get out of the bed. It is currently difficult to use a bed pan and there are no good means available to wash and dry the patient.

### BRIEF SUMMARY OF THE INVENTION

The present invention relates to a waste management system comprising a bedpan which is coupled with a system for providing air, suction and water to the bedpan and positioned to receive bodily waste from a user. The air and water are directed onto genitals, perineum, and/or an anus of the user to rinse and dry these areas after a bowel movement. The suction connection is posited to remove bodily waste. There are no electronic parts and the air suction and water are provided from hospital sources usually found in hospital rooms as well as by manual means.

Accordingly, in one embodiment, there is a human waste capturing device comprising:
a) a shell comprising:
   i. a first end having a wedge-shaped flap designed to be placed between a mattress and the buttocks of a user;
   ii. a second end having a receptacle for inserting a liner which is adjustable relative to the first end wedge;
   iii. a waste tube; and
   iv. an inlet for each of water and air;
b) a liner for placement in the shell receptacle; and
c) a waste disposal container in fluid communication with the shell waste tube waste disposal container having a connector inlet for a suction tube.

### BRIEF DESCRIPTION OF THE DRAWINGS

FIG. 1 is a perspective view of the second end.

FIG. 2 is an exploded view of the liner and the second end.

FIG. 3 is a perspective view of a user in a hospital bed.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publication may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

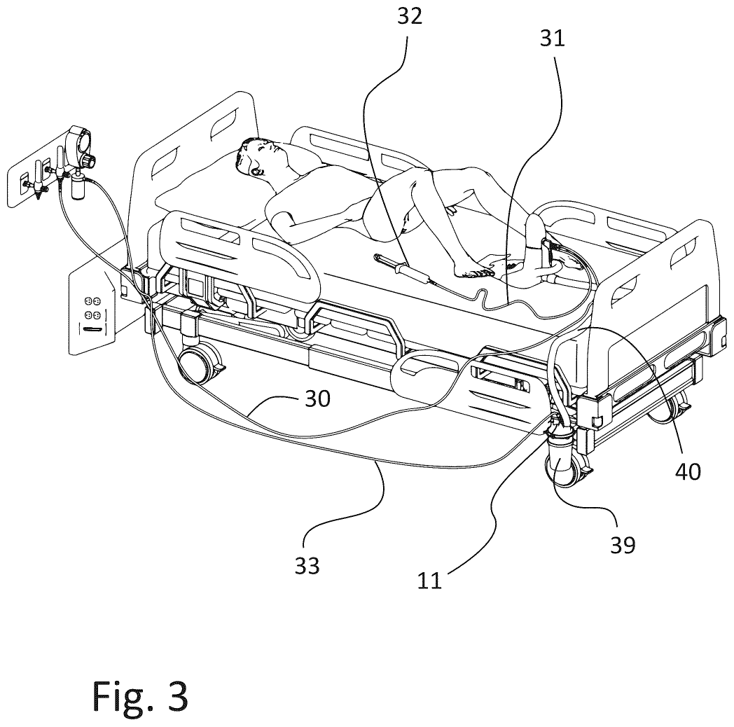

As used herein, the term “human waste capturing device” refers to a device for capturing human waste, both urine and feces, while a human is in the prone position. Typically, these devices can be called bedpans. The capturing device of the present invention is designed to remove human waste from a user as well as rinse and dry at least one of the genitals, the perineum, and the anus of the user. External sources of water, air and suction are used to provide the desired result. Suction is used in the waste disposal container to remove the human waste to the container. The air, water and suction are provided by external sources such as are available in most hospital settings. Other manual ways of providing air, water and suction can be utilized. In the drawing of FIG. 3 a syringe is utilized to provide water.

As used herein, the term “shell” refers to the basic structure of the device, it also is designed to accept a removable liner to collect the human waste. It comprises a first end flap, a second end for inserting the liner, a waste tube, an air and water inlet. The waste tube is connected in fluid communication with a waste disposal container, and waste is removed from the waste tube via suction applied to the waste disposal container.

As used herein, the term “first end” of the shell refers to a wedge-shaped flap designed to be placed between the mattress and the buttocks of the user. It can be made of a compression material and elastomer or a high-density nylon, or polyester fabric, or the like.

As used herein, the term “wedge shaped flap” refers to the flap positioned at a first end of the shell for user placement of the shell flap between the mattress and buttocks of the user. It can be made of a compression material and elastomer or a high-density nylon, or polyester fabric, or the like.

As used herein, the term “second end” refers to the end of the shell which has a receptacle for inserting a liner, it also has a waste tube outlet for removal of the waste and inlets for each of water and air. The inlets can be connected to spray nozzles and they can be height adjusted or removed, positioned to direct the water and air to at least one of the genitals, the perineum, and the anus of the user for the purpose of rinsing and drying.

As used herein, the term “liner” refers to a removable container for receiving human waste. It is positioned in the second end receptacle for inserting the liner.

As used herein, the term “a waste disposal container” refers to a container in fluid communication with the waste tube of the shell. Waste is drawn into the container by attaching suction tube to the suction connector inlet.

As used herein, the term “connector inlet for a suction tube” refers to an inlet for attaching a hose supplying suction to the container in order to draw the human waste into the container. Suction is applied from an independent source attached to the inlet.

As used herein the term “inlet” refers to a water and air inlet designed to bring water for cleansing and air for drying after a bowel movement. In one embodiment, the air and water inlet are height-adjustable.

### DRAWINGS

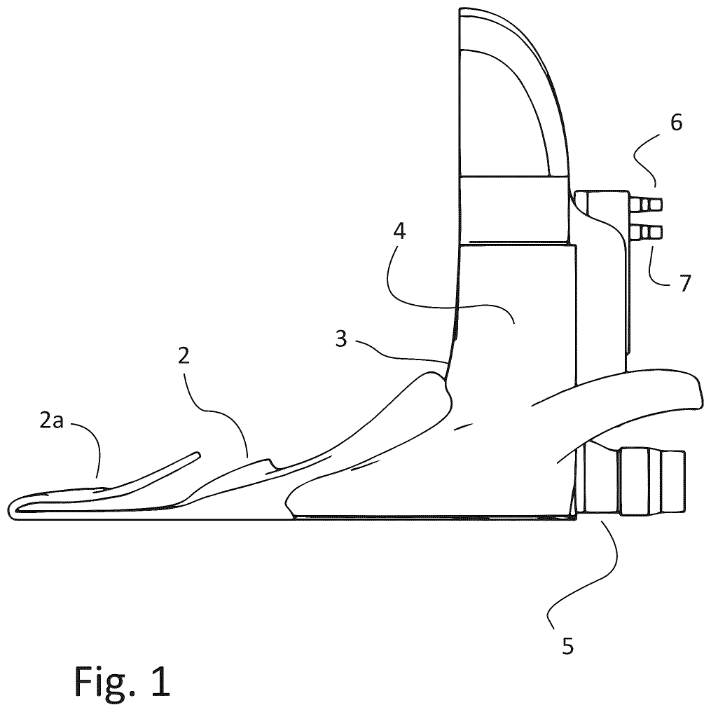

Now referring to the drawings, FIG. 1 is a perspective view of the shell of the invention. In this view a first end 2 having a wedge-shaped flap 2a designed to be placed between a mattress and the buttocks of a user. A second end 4 having a receptacle 3 for inserting a liner which is adjustable relative to the first end wedge. Waste tube 5 delivers the waste to a waste container not shown in this figure by means of suction. Air inlet 6 and water inlet 7 allow connection of air and water lines. In the present view, the device holding the two inlets is removable or adjustable in height.

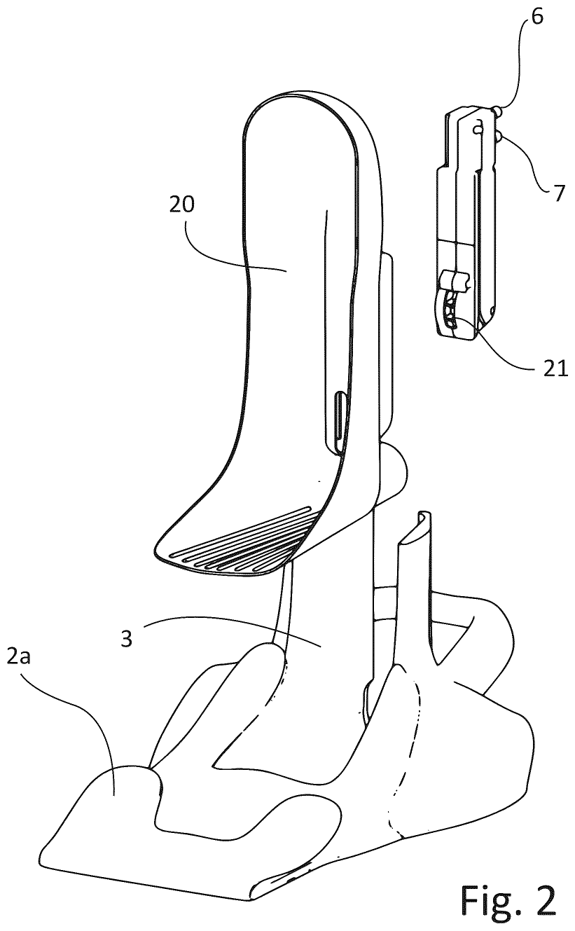

FIG. 2 is an exploded view of the shell plus removable liner 20. An air inlet 6 and water inlet 7 are shown as removable. In this view the nozzles 21 as shown for spaying water or air to the user are connected to the two nozzles respectively.

FIG. 3 shows the entire device, used with prone patient 38, connected to air line 30 and water line 31. In this view water is supplied by use of a syringe 32, while air and suction lines 33 are provided in a room, such as a hospital acute care facility, and the like. Shown in this view is the waste container 39 connected to the shell waste tube via waste connecting tube 40. Vacuum line 33 is connected to waste container inlet 41 for providing vacuum to draw the waste to the container.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A human waste capturing device comprising:
a) a shell comprising:
i. a first end having a wedge-shaped flap designed to be placed between a mattress and the buttocks of a user;
ii. a second end having a receptacle for inserting a liner which is adjustable relative to the first end wedge;
iii. a waste tube; and
iv. an inlet for each of water and air;
b) a liner for placement in the shell receptacle; and
c) a waste disposal container in fluid communication with the shell waste tube waste disposal container having a connector inlet for a suction tube attachment.

2. The human waste capturing device according to claim 1 wherein the inlet for water has a spray nozzle positioned to direct water to at least one of the genitals, the perineum, and the anus of the user.

3. The human waste capturing device according to claim 1 wherein the inlet for air has a nozzle connected to the air inlet positioned to direct air to at least one of the genitals, the perineum, and/or the anus of the user.

4. The human waste capturing device according to claim 1 or claim 3 wherein the flap is configured to shield the water and air nozzles from human waste.

5. The human waste capturing device according to claim 1 wherein the flap comprises a compressible material.
